(19) United States
(12) Patent Application Publication
(10) Pub. No.: US 2025/0009816 A1
(43) Pub. Date: Jan. 9, 2025

(54) Engineered probiotic platform for in-situ conversion of sugars to functional ingredients

(71) Applicant: GutMind Corp, Chapel Hill, NC (US)

(72) Inventor: Akbar Farzin Javan, Chapel Hill, NC (US)

(73) Assignee: Gutmind Corp, Chapel Hill, NC (US)

(21) Appl. No.: 18/219,649

(22) Filed: Jul. 8, 2023

(51) Int. Cl. A61K 35/745; A61K 35/742; A61K 35/747

(52) U.S. Cl. CPC A61K 35/745; A61K 35/742; A61K 35/747; A61K 2035/115

(57) ABSTRACT

An engineered probiotics platform for in-situ conversion of simple sugars, which are consumed at an unhealthy level in the Western diet, to key functional ingredients such as omega-3 fatty acids, which are critically lacking in the Western diet.

### FIELD OF THE INVENTION

The present invention relates to the use of an engineered probiotic platform to convert sugars, which are consumed at an unhealthy level in the Western diet, to key functional ingredients such as omega-3 fatty acids, which are critically lacking in the Western diet.

### BACKGROUND OF THE INVENTION

Sustainable changes to the gut microbiome require sustainable changes to our diets, and only 5% of Americans consume the recommended daily fiber intake in their diets to benefit from key metabolites produced by good bacteria (the “fiber gap”). Overconsumption of sugar further exacerbates the problem.

Additionally, only 10% of adults in America consume their recommended daily serving of vegetables, and just 12% get enough fruit. The story is the same in much of the world. As such, many turn to vitamin and mineral supplements to make up for their dietary deficiencies. In 2018, 54% of North Americans were taking a nutritional supplement. So, there is a whopping 40% of Americans don't get sufficient key functional ingredients that are critical for mental and physical health through food or supplements.

Our solution is to leverage an engineered probiotic platform to convert excess sugar into key functional ingredients that are missing in the Western diet. In much the same way gut bacteria produce some of our needed vitamins, we would like to leverage our engineered probiotics to convert excess sugar into healthier functional ingredients in a similar fashion.

Average Americans consume about 90 grams of added sugar daily; each 12 oz of soda has about 35-40 grams of sugar. Our first target is an engineered probiotic that metabolizes sugar to EPA or DHA. As many as 95% of Americans do not get enough DHA and EPA. Assuming 50% of the theoretical yield, we would need to convert only about 4% of the sugar from a 12 oz can of soda to close the “omega 3 gap”.

With advancements in synthetic biology, many metabolites which are produced through biosynthesis, can be programmed in microbial hosts, including probiotic bacteria. These advancements have paved the way for use of probiotics for in-situ delivery of drugs or metabolization of harmful compounds.

### SUMMARY OF THE INVENTION

The present invention is an engineered probiotic which can metabolize sugars and produce DHA or EPA in-situ in the human gut system. This invention addresses two key health challenges: 1) Excess sugars consumed at an unhealthy level in the Western diet, which is a precursor to type 2 diabetes, a fatty liver, and cardiovascular disease, and 2) Lack of diversity of ingredients, and thus certain key functional ingredients in Western diet, which are required for physical- and mental-health.

This invention is also comprised of engineering a probiotic bacterium to serve as a platform to metabolize sugars and convert them into other key functional ingredients such as short-chain fatty acids (butyric and propionic acids), capsaicin (known for its weight loss benefits), or anti-inflammatory compounds (such as curcumin). The target list could include any ingredients that are viable through biosynthesis. Additionally, an engineered probiotic could be designed to simply lower calories from sugar via fatty acid production and storage, which leave the body undigested.

### DETAILED DESCRIPTION OF THE INVENTION

The invention is comprised of engineering the functional ingredients such EPA or DHA biosynthesis pathway into a probiotic bacterium such as Lactobacillus plantarum, Lactobacillus rhamnosus, Lactobacillus reuteri, Bifidobacterium animalis, Bifidobacterium lactis, and Clostridium spp which have competitive sugar uptake machinery. The biosynthesis pathways for functional ingredients such as EPA and DHA have been already engineered in a number of microbial hosts, including E. coli and L. lactis.

In a preferred embodiment, the production of functional ingredients is triggered by a pH inducible gene expression for production of the functional ingredient in a targeted location in the gut system. This design enables the production of probiotics during fermentation at a different pH to prioritize the production of probiotic cells vs the targeted functional ingredients.

In a preferred embodiment, the engineered probiotic is evolved to survive or thrive better in the harsh gut environment, but have slow reproduction rate, so it does not entrench in the gut system.

1. An engineered probiotic, including but not limited to Lactobacillus plantarum, Lactobacillus rhamnosus, Lactobacillus reuteri, Bifidobacterium animalis, Bifidobacterium lactis, and Clostridium spp, for in-situ (in the gut system) uptake of simple sugars

2. The method of claim 1, wherein the engineered probiotic converts simple sugars to key functional ingredients.

3. The method of claims 1 and 2, wherein the key functional ingredient is omega 3 fatty acids.

4. The method of claims 1, 2 and 3, wherein the omega 3 fatty acids are EPA or DHA.

5. The method of claims 1 and 2, wherein the target functional ingredients are short-chain fatty acids (i.e. butyric and propionic acids), weight loss beneficial compounds (e.g. capsaicin), or anti-inflammatory compounds (such as curcumin).

6. The method of claim 1, wherein the engineered probiotic uptakes sugars, and converts them to fatty acids but doesn't excrete the fatty acids. This is simply to lower calories from sugars via fatty acid production and storage by the engineered probiotic cells, which leave the body undigested.
